SEQUENCE LISTING (2 sequences)

SEQ ID NO: 1 - protein, 161 aa, Homo sapiens
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIEN

SEQ ID NO: 2 - protein, 235 aa, Homo sapiens
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

(19) United States
(12) Patent Application Publication
(10) Pub. No.: US 2007/0099266 A1
(43) Pub. Date: May 3, 2007

(54) Process for the production of tumor necrosis factor-binding proteins

(75) Inventor: Yolande Rouiller, Epalinges (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Applied Research Systems ARS Holding N.V., Curacao (NL)

(21) Appl. No.: 10/582,952

(22) PCT Filed: Dec. 21, 2004
(86) PCT No.: PCT/EP04/53642

§ 371 Date: Jun. 15, 2006

(30) Foreign Application Priority Data
Dec. 23, 2003 (EP) 03104958.8

(51) Int. Cl. C07K 14/715; C07H 21/04; C12P 21/06

(52) U.S. Cl. 435/69.1; 435/320.1; 435/325; 530/350; 536/23.5

(57) ABSTRACT

The invention provides methods for increasing the recombinant production of polypeptides, in particular Tumor Necrosis Factor Binding Proteins, from mammalian cells at a temperature below 30° C.

### FIELD OF THE INVENTION

The invention is in the field of recombinant production of polypeptides, particularly of TNF binding proteins, from mammalian cells.

### BACKGROUND OF THE INVENTION

Mammalian cell lines are widely used in Biotechnology to produce therapeutically important proteins such as monoclonal antibodies, cytokines, growth factors and coagulation factors. Among the various parameters responsible for an optimised process leading to a high yield of active product, the cell cycle phase in which the producing cells are, might play an important role. If initial cell growth is essential to get enough cells for production, cell proliferation beyond a certain density might induce the accumulation of waste products and cell death (Goldman et al., 1997; Munzert et al., 1996). Low temperature cultivation is one of the strategies enabling to control cell proliferation (Moore et al., 1997; Kaufmann et al., 1999). Temperatures below 37° C. have been reported to affect other cellular events, such as decreasing glucose consumption, lactate production and extending cell viability probably by delaying the onset of apoptosis (Chuppa et al., 1997; Furukawa and Ohsuye, 1998; Moore et al., 1997; Weidemann et al., 1994).

The effects of low cultivation temperatures on the protein production depend on a variety of parameters such as cell lines or promoters used (Barnabe and Butler, 1994; Chuppa et al., 1997; Furukawa and Ohsuye, 1998; Furukawa and Ohsuye, 1999; Kaufmann et al., 1999; Sureshkumar and Mutharasan, 1991; Weidemann et al., 1994). For example, temperatures below 37° C. decreased monoclonal antibody production by hybridoma cells (Barnabé and Butler, 1994; Sureshkumar and Mutharasan, 1991). Such temperatures did not affect Antithrombin III production by BHK cells (Weidemann et al., 1994) while they increased the specific productivity of recombinant CHO cells producing secreted alkaline phosphatase (Kaufmann et al., 1999), α-amidating enzyme (Furukawa and Ohsuye, 1999; Furukawa and Ohsuye, 1998), tissue plasminogen activator (Hendrick et al., 2003) or erythropoietin (Yoon et al., 2003).

Many of the recombinant proteins developed for human therapeutics are glycoproteins expressed in mammalian cells, such as for example erythropoietin, interleukin-2, interferon-β, immunoglobulins or tissue plasminogen activator. Carbohydrate components of glycoproteins can play a crucial role in protein solubility, stability, bioactivity, immunogenicity and clearance from the blood stream (Jenkins et al., 1996). The N-linked glycosylation pathway starts with the synthesis of a lipid-linked oligosaccharide and is followed by the co-translational transfer of the oligosaccharide to a specific asparagine residue on the nascent polypeptide in the endoplasmic reticulum and by subsequent monosaccharide changes as the protein passes through the endoplasmic reticulum and Golgi apparatus (Hirschberg and Snider, 1987). As the transfer of the oligosaccharide precursor does not always proceed to completion, a given protein might be produced as a heterogeneous mixture of differently glycosylated products. The extent of glycosylation might have an influence on the quality of the recombinant protein; therefore, it is an important parameter to consider for producing a therapeutic product of consistent quality.

Glycosylation, as other post-translational modifications, e.g. phosphorylation and methylation, have been shown to depend on the enzymatic machinery of the host cells and culture conditions (Gawlitzek et al., 2000; Jenkins et al., 1996; Kaufmann et al., 2001; Nyberg et al., 1999). Among the cell culture factors tested, ammonia, protein and lipid content of the medium, pH, and culture length, have been shown to affect glycosylation (Yang and Butler, 2000; Werner et al., 1998; Castro et al., 1995; Borys et al., 1993; Andersen et al., 2000). Other studies suggest that the oligosaccharide profile of glycoproteins varies depending on the proliferation rate of cells. Kaufmann et al, while comparing the glycosylation profiles of secreted alkaline phosphatase (SEAP) produced by proliferating versus growth controlled CHO cells, showed an effect on the oligosaccharide profile of glycoproteins of SEAP when CHO proliferation was carried out at a low temperature while there was no effect when the proliferation was controlled by an over expression of the cyclin-dependent kinase inhibitor p27 (Kaufmann et al., 2001). The low temperature increased the disialylated glycoform fraction from 70 to 80%. Andersen et al described an increase in glycosylation site occupancy at Asn-184 of human tissue plasminogen activator (t-PA) produced in recombinant CHO cells at 33° C. versus 37° C. (Andersen et al., 2000). A moderately higher overall sialylation was observed in the glycosylated pattern of erythropoietin (EPO) synthesized by BHK cells, whose growth was inhibited by the transcription factor IRF-1 (Mueller et al., 1999), when compared to proliferating cells.

U.S. Pat. No. 5,705,364 describes preparation of glycoproteins in mammalian cell culture wherein the sialic acid content of the glycoprotein produced was controlled over a broad range of values by manipulating the cell culture environment, including the temperature. The host cell was cultured in a production phase of the culture by adding an alkanoic acid or salt thereof to the culture at a certain concentration range, maintaining the osmolality of the culture at about 250 to about 600 mOsm, and maintaining the temperature of the culture between 30° C. and 35° C.

In a further previous study, Ducommun et al (Ducommun et al., 2002) showed that lowering the temperature from 37° C. to 33.5 and then 32° C. in a packed bed bioreactor process containing recombinant CHO cells enabled to increase the specific production rate of the protein of interest by a factor of six when compared to a permanent state at 37° C.

WO0036092 provides methods for the expression of high yields of IgG fused to a TNF family receptor member (LTβR) by culturing transformed hosts at a low temperature, about 27° C. to 32° C., minimizing thereby the amount of misfolded protein forms.

EP0764719 provides methods for improving productivity of cultured cells comprising the steps of culturing the cells at a temperature allowing cell growth and then culturing the animal cells at a temperature of 30 to 35° C.

WO03/083066 provides a method for producing a recombinant polypeptide comprising culturing a mammalian cell line in a growth phase followed by a production phase which can occur at a temperature of less than 37° C. (from 29° C. to about 36° C.) adding into the culture medium during the production phase a xanthine derivative in order to increase the production.

An increase of production of TNFR:Fc, i.e. Fc portion of an antibody fused to an extracellular domain of TNFR or RANK:FC, i.e. Fc portion of an antibody fused to an extracellular domain of a Type I transmembrane protein member of the TNF receptor superfamily RANK (receptor activator of NF-KB), was shown in CHO cells at a minimum temperature of 31° C. in the presence of increasing amounts of inducers (xanthine derivatives such as caffeine).

Tumor necrosis factor-alpha (TNFα, TNF-alpha), a potent cytokine, elicits a broad spectrum of biologic responses that are mediated by binding to a cell surface receptor.

TNF-alpha has been shown to be involved in several diseases, examples of which are adult respiratory distress syndrome, pulmonary fibrosis, malaria, infectious hepatitis, tuberculosis, inflammatory bowel disease, septic shock, AIDS, graft-versus host reaction, autoimmune diseases, such as rheumatoid arthritis, multiple sclerosis and juvenile diabetes, and skin delayed type hypersensitivity disorders. The intracellular signals for the response to TNF-alpha are provided by cell surface receptors (herein after TNF-R), of two distinct molecular species, to which TNF-alpha binds at high affinity.

The cell surface TNF-Rs are expressed in many cells of the body. The various effects of TN F-alpha, the cytotoxic, growth promoting and others, are all signalled by the TNF receptors upon the binding of TNF-alpha to them. Two forms of these receptors, which differ in molecular size, 55 and 75 kilodaltons, have been described.

Both receptors for TNF-alpha exist not only in cell-bound, but also in soluble forms, consisting of the cleaved extracellular domains of the intact receptors, in situ derived by proteolytic cleavage from the cell surface forms. These soluble TNF-alpha receptors (sTNF-Rs) can maintain the ability to bind TNF-alpha and thus compete for TNF-alpha with the cell surface receptors and blocking thereby TNF-alpha activity. These soluble TNF alpha receptors are also known as TBPs (TNF binding proteins).

The potential therapeutic actions of TNF binding proteins are in general related to their ability to neutralize the detrimental effects of an accumulation of high concentrations of TNF in the body.

TNF alpha Receptor I is also known as TNFAR (Tumor Necrosis Factor-Alpha Receptor), TNFR1 (Tumor Necrosis Factor Receptor 1), TNFR55, TNFR60 and TNFRSF1A (Tumor Necrosis Factor Receptor Superfamily, Member 1 a). Its cDNA has been cloned and its nucleic acid sequence determined (see Loetscher et al., 1990; Nophar et al., 1990; Smith et al., 1990).

The term “TBP-1”, TNF binding protein 1, as used herein, relates to the extracellular, soluble fragment of human TNF Receptor-1 (p55 sTNF-R), comprising the amino acid sequence corresponding to the 20-180 amino acids fragment of Nophar et al. (Nophar et al., 1990). The International Non-proprietary Name (INN) of this protein is “onercept”.

Onercept in being developed for the potential treatment of a number of disorders including reperfusion injury, male infertility, endometriosis, inflammation, multiple sclerosis, plasmodium infection, psoriasis, rheumatoid arthritis, autoimmune diseases, cachexia, transplant rejection, septic shock and Crohn's disease.

TNF alpha Receptor II is also known as TNFRSF1 B (Tumor Necrosis Factor Receptor Subfamily, Member 1b), TNFR2 (Tumor Necrosis Factor Receptor 2), TNFBR (Tumor Necrosis Factor, Beta Receptor), TNFR75 and TNFR80. Schall et al. isolated a cDNA corresponding to TNFR2 using oligomer probes based on amino acid sequence from the purified protein (Schall et al., 1990). The receptor encodes a 415-amino acid polypeptide with a single membrane-spanning domain and has an extracellular domain with sequence similarity to nerve growth factor receptor and B-cell activation protein Bp50.

The term “TBP-2”, TNF binding protein 2, as used herein, relates to the extracellular, soluble fragment of human TNF Receptor-2 (p75 sTNF-R), comprising the amino acid sequence corresponding to the 1-235 amino acids fragment of the full-length receptor.

For development and commercialisation of polypeptide-based drugs, high amounts of the polypeptide are required. Therefore, there is a need to continually improve yields of recombinant polypeptides without altering the quality of the polypeptide, e.g. in terms of glycosylation regarding the most abundant species.

### SUMMARY OF THE INVENTION

The present invention is based on the elucidation of the optimal productivity temperature for TBP-1 by CHO cells in a range of temperatures from 37 to 25° C. This series of experiments showed that a production phase carried out at a temperature of below 29° C. resulted in highly improved yields of TBP-1 without altering its quality in terms of glycosylation.

Therefore it is the first object of the invention to provide a method for producing a recombinant polypeptide comprising culturing a mammalian cell line, which expresses a recombinant polypeptide, in a production phase at a temperature below 29° C, the polypeptide being preferably a Tumor Necrosis Factor Binding Protein (TBP).

A second aspect of the invention relates to the use of a temperature of 24 or 25 or 26 or 27 or 28 or 29° C. for the production of a protein.

In a third aspect of the invention, the polypeptide obtained, is mono-glycosylated.

The fourth aspect of the invention relates to a composition comprising a mixture of a mono-glycosylated protein and its bi- glycosylated and tri-glycosylated forms.

### BRIEF DESCRIPTION OF THE DRAWINGS

FIG. 1 shows glucose consumption and lactate production of the different cultures at 25° C., 29° C., 32° C., 34° C. and 37° C.

FIG. 2 shows the amount of TBP-1 secreted per ml of medium tested at each temperature (25° C., 29° C., 32° C., 34° C. and 37° C. Titers were normalized by setting the maximum value to 100.

FIG. 3 shows specific productivity of the TBP-1 at different temperatures. Specific productivity in pcd (picogram per cell and per day) was normalized by setting the maximum value 20 to 100. Two separate experiments (Exp 1 and Exp 2), performed under the same conditions, are shown.

FIG. 4 shows glucose and lactate concentrations as a function of time, in high (4 g/L) and standard (2.5 g/L) glucose culture medium.

FIG. 5 shows titers of the TBP-1 as a function of time, in high (4 g/L) and standard glucose (2.5 g/L) culture medium. Titers were normalized by setting the maximum value to 100. HG=high glucose.

FIG. 6 shows specific productivity of the TBP-1 as a function of time, in high (4 g/L) and standard (2.5 g/L) glucose. Titers were normalized by setting the maximum value to 100. HG=high glucose.

FIG. 7 shows Mass Spectrometry (MS) profiles as a function of temperature. 0=0 sialic acid; 1=1 sialic acid; 2=2 sialic acid; 3=3 sialic acid; 4=4 sialic acid.

FIG. 8 shows Mass Spectrometry (MS) profiles from samples obtained from standard (2.5 g/L) and high (4 g/L) glucose cell culture media. HG=high glucose.

FIG. 9 shows titers of TBP-1 at different production temperatures during fed-batch development at 5L scale. TBP-1 normalized titers are shown from day 6 to 24 at 29° C. (run 1), at 31° C. (run 2) and 34° C. (run 3).

### DETAILED DESCRIPTION OF THE INVENTION

In the frame of the present invention it has been found that lowering the temperature from 37° C. to at or below 29° C. had a beneficial effect on the productivity of recombinant CHO cells, increasing the amount of a secreted glycoprotein, in particular TBP-1, more than 10 fold without considerably altering its quality in terms of glycosylation regarding the most abundant species (bi-glycosylated bi-antennary).

Therefore the invention relates to a method for producing a recombinant polypeptide comprising culturing a mammalian cell line, the cell line expressing a recombinant polypeptide, in a production phase at a temperature at or below 29° C.

In the context of the present invention the expressions “cell”, “cell line”, and “cell culture” are used interchangeably, and all such designations include progeny.

The term “production phase” means a period during which cells are producing high amounts of recombinant polypeptide. A production phase is characterized by a lower cell division than during a growth phase and by the use of medium and culture conditions designed to maximize polypeptide production.

Preferably the invention relates to a method for producing human TNF binding proteins (TBP) and most preferably recombinant human TBP-1 or TBP-2, or a mutein, salt, isoform, fused protein, functional derivative, active fraction thereof.

The term “TBP-1”, TNF binding protein 1, as used herein, relates to the extracellular, soluble fragment of human TNF Receptor-1, comprising the amino acid sequence corresponding to the 20-180 amino acids fragment of Nophar et al. (Nophar et al., 1990), whose International Non-proprietary Name (INN) is “onercept”. The sequence of human TBP-1 is reported herein as SEQ ID NO: 1 of the annexed sequence listing.

The term “TBP-2”, TNF binding protein 2, as used herein, relates to the extracellular, soluble fragment of human TNF Receptor-2 (p75 sTNF-R), comprising the amino acid sequence corresponding to the 1-235 amino acids fragment (Smith et al., 1990). The sequence of human TBP-2 is reported herein as SEQ ID NO: 2 of the annexed sequence listing.

In a preferred embodiment the mammalian cell comprises a DNA sequence coding for TBP-1 selected from the group consisting of
(a) A polypeptide comprising SEQ ID NO: 1;
(b) A mutein of (a), wherein the amino acid sequence has at least 40% or 50% or 60% or 70% or 80% or 90% identity to the sequence in (a);
(h) A mutein of (a) which is encoded by a DNA sequence, which hybridizes to the complement of the native DNA sequence encoding (a) under moderately stringent conditions or under highly stringent conditions;
(i) A mutein of (a) wherein any changes in the amino acid sequence are conservative amino acid substitutions to the amino acid sequences in (a);
(j) A salt or an isoform, fused protein, functional derivative, active fraction or circularly permutated derivative of (a).

In a further preferred embodiment the mammalian cell line comprises a DNA sequence coding for TBP-2 selected from the group consisting of
(a) A polypeptide comprising SEQ ID NO: 2;
(b) A mutein of (a), wherein the amino acid sequence has at least 40% or 50% or 60% or 70% or 80% or 90% identity to the sequence in (a);
(h) A mutein of (a) which is encoded by a DNA sequence, which hybridizes to the complement of the native DNA sequence encoding (a) under moderately stringent conditions or under highly stringent conditions;
(i) A mutein of (a) wherein any changes in the amino acid sequence are conservative amino acid substitutions to the amino acid sequences in (a);
(j) A salt or an isoform, fused protein, functional derivative, active fraction or circularly permutated derivative of (a).

As used herein the term “muteins” refers to analogs of TBP-1 or TBP-2, in which one or more of the amino acid residues of a natural TBP-1 or TBP-2 are replaced by different amino acid residues, or are deleted, or one or more amino acid residues are added to the natural sequence of TBP-1 or TBP-2, without changing considerably the activity of the resulting products as compared to the wild-type TBP-1 or TBP-2. These muteins are prepared by known synthesis and/or by site-directed mutagenesis techniques, or any other known technique suitable therefore. In the frame if the present invention the term “mutein” does not encompass Immunoglobulin (Ig) fusion proteins.

Muteins of TBP-1 or TBP-2, which can be used in accordance with the present invention, or nucleic acid coding thereof, include a finite set of substantially corresponding sequences as substitution peptides or polynucleotides which can be routinely obtained by one of ordinary skill in the art, without undue experimentation, based on the teachings and guidance presented herein.

Muteins in accordance with the present invention include proteins encoded by a nucleic acid, such as DNA or RNA, which hybridizes to DNA or RNA, which encodes TBP-1 or TBP-2, in accordance with the present invention, under moderately or highly stringent conditions. The term “stringent conditions” refers to hybridization and subsequent washing conditions, which those of ordinary skill in the art conventionally refer to as “stringent”.

See Ausubel et al., Current Protocols in Molecular Biology, supra, lnterscience, N.Y., §§6.3 and 6.4 (1987, 1992), and Sambrook et al. (Sambrook, J. C., Fritsch, E. F., and Maniatis, T. (1989) Molecular Cloning: A Laboratory Manual, Cold Spring Harbor Laboratory Press, Cold Spring Harbor, N.Y.).

Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1−SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC. See Ausubel, supra.

In a preferred embodiment, any such mutein has at least 40% identity or homology with the sequence of SEQ ID NO: 1 or 2 of the annexed sequence listing. More preferably, it has at least 50%, at least 60%, at least 70%, at least 80% or, most preferably, at least 90% identity or homology thereto.

Identity reflects a relationship between two or more polypeptide sequences or two or more polynucleotide sequences, determined by comparing the sequences. In general, identity refers to an exact nucleotide-to-nucleotide or amino acid-to-amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9 (Devereux et al., 1984), for example the programs BESTFIT and GAP, may be used to determine the % identity between two polynucleotides and the % identity and the % homology between two polypeptide sequences. BESTFIT uses the “local homology” algorithm of Smith and Waterman (Smith and Waterman, 1981) and finds the best single region of similarity between two sequences. Other programs for determining identity and/or similarity between sequences are also known in the art, for instance the BLAST family of programs (Altschul et al., 1990; Altschul et al., 1997), accessible through the home page of the NCBI at www.ncbi.nim.nih.gov) and FASTA (Pearson, 1990; Pearson and Lipman, 1988).

Preferred changes for muteins in accordance with the present invention are what are known as “conservative” substitutions. Conservative amino acid substitutions of TBP-1 or TBP-2 polypeptides, may include synonymous amino acids within a group which have sufficiently similar physicochemical properties that substitution between members of the group will preserve the biological function of the molecule (Grantham, 1974; Pearson, 1990; Pearson, 1990). It is clear that insertions and deletions of amino acids may also be made in the above-defined sequences without altering their function, particularly if the insertions or deletions only involve a few amino acids, e.g. under thirty, and preferably under ten, and do not remove or displace amino acids which are critical to a functional conformation, e.g. cysteine residues. Proteins and muteins produced by such deletions and/or insertions come within the purview of the present invention.

A “fragment” of TBP-1 or TBP-2 according to the present invention refers to any subset of the molecule, that is, a shorter peptide, which retains the desired biological activity.

It was found in the frame of the present invention that glucose was metabolized much faster at 37° C., 34° C. and 32° C. than at lower temperatures (29 and 25° C.) and its consumption was nearly complete after 4 days of culture for the temperatures above 30° C. This decrease in glucose was correlated with an increase in lactate production at the higher temperatures.

Specific productivity increased with decreasing temperatures and was optimal at 29° C. with an increase of more than ten fold in comparison to that obtained at 37° C. The low productivity at 37° C. was not due to a depletion of glucose in the culture, as shown by the absence of increase in productivity with a higher glucose concentration in the culture medium.

Therefore, in a preferred embodiment the mammalian cell is cultured at a temperature between 20° C. and 29° C. The cells may be cultured at about 20, 21, 22, 23, 24, 25, 26, 27, 28 or 29° C. More preferably, the method of the invention is carried out at a temperature of about 25 to 29° C.

In a further preferred embodiment the mammalian cell is cultured at a temperature of about 26° C., or about 27° C., or about 28° C.

It is highly preferred that the mammalian cell be cultured at a temperature of about 29° C.

The method according to the invention may be carried out in any mammalian cell expressing system. Preferably, the mammalian cell line according to the invention is VERO, HeLa, 3T3, CV1, MDCK, BHK, Human Kidney 293, and more preferably a CHO cell line. A human cell line, such as Human Kidney 293, may also be cultured in accordance with the present invention.

In a preferred embodiment of the invention the medium used during the production phase is serum free.

The cell culture medium is generally “serum free” when the medium is essentially free of compounds from any mammalian source (such as e.g. foetal bovine serum (FBS)) and includes the minimal essential substances required for cell growth. By “essentially free” is meant that the cell culture medium comprises between about 0-5% serum, preferably between about 0-1% serum, and most preferably between about 0-0.1% serum. Advantageously, serum-free chemically “defined” medium can be used, wherein the identity and concentration of each of the components in the medium is known (i.e., an undefined component such as bovine pituitary extract (BPE) is not present in the culture medium). This type of medium avoids the presence of extraneous substances that may affect cell proliferation or unwanted activation of cells.

The invention further relates to a process for collection or recovery of the polypeptide from the medium.

Preferably, the method further comprises the step of purifying the polypeptide from any unwanted medium or cell derived components.

The invention further comprises formulating the purified polypeptide with a pharmaceutically acceptable carrier. The formulation is preferably for human administration.

The definition of “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

Another aspect of the invention relates to the use of a temperature of 24, 25, 26, 27, 28 or preferably 29° C. for the production of a protein.

TBP-1 is a glycoprotein with three putative complex type N-linked glycosylation sites on asparagine residues, the main isoforms corresponding to molecules with two glycosylation sites occupied. Protein glycosylation may significantly alter protein properties and since the glycosylation pattern can vary with changes of culture conditions, the quality of TBP-1 secreted under the various temperature conditions was analysed in terms of glycosylation using mass spectrometry. It was found that the glycosylation of the molecule, with regard to the proportion of the most abundant species, i.e. bi-glycosylated bi-antennary, is comparable at all temperatures tested and is not affected by the concentration of glucose in the medium.

At the lower temperatures however, the proportion of some minor forms, such as partially glycosylated species, increased. These findings were confirmed by S-index, an indicator of the overall sialylation level of a protein calculated from the raw data spectrum from mass spectrometry (MALDI-TOF) considering the relative intensities of the ions of the main oligosaccharide species.

Therefore, another aspect of the invention relates to polypeptide obtainable according to the above-described processes, the polypeptide being preferably mono-glycosylated. The inventors of the present invention have for the first time identified a cell culture method for the production of mono-glycosylated TBP-1. Preferably the polypeptides of the invention have an S-Index above 195, preferably above 200, preferably above 200, preferably above 250, preferably above 260 or preferably above 265.

The invention further relates to a composition comprising a combination of mono-, bi- and tri-glycosylated forms of a polypeptide. The polypeptide is preferably recombinant human TBP-1.

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLES

Materials and Methods

The cell line used in the following experiments is Chinese hamster ovary (CHO) cell line genetically engineered to secrete recombinant TBP-1 (Laboratoires Serono S. A., Corsier-sur-Vevey, Switzerland). The cells were cultured in a serum-free medium containing 2.5 g/L or 4.0 g/L of glucose.

Culture in Tissue Culture Flasks (TCF)

After expansion in cell culture medium at 37° C., cells were centrifuged and re-suspended in fresh medium at a concentration of 0.6×10⁶ cells/ml. Cells were then transferred into tissue culture flasks (TCF: Corning, 25 and 175 cm²) and the cultures were performed in batch-mode in a humidified atmosphere of 5% CO₂ in air at 25, 29, 32, 34 and 37° C. The working volume was 10 ml for TCF25 and 120 m/l for TCF175.

Culture in 5L Bioreactors

Cells were grown in a 5L nominal volume bioreactor with a maximum working volume of 3.5 L (Celligen Plus, New Brunswick Scientific, Edison, USA) in a fed-batch mode. The growth phase was performed at 37° C. and the production phase started after a switch of the temperature to 34, 31 or 29° C.

### Example 1

### Cell Density and Metabolic Assays

Eperimental Design

Experiments for the determination of cell metabolic activities were performed in TCF25. Seven replicates were incubated at each temperature, and every day during 7 days, one TCF of each temperature was tested for cell density, viability, glucose consumption, lactate production and productivity.

Cell density and metabolic assays (glucose, lactate, productivity) were performed daily. Cell counts were performed using the Trypan blue exclusion method (0.4% Sigma). Glucose and lactate concentrations were determined on filtered (0.8/0.2 μm filter, Gelman) aliquots using an EML 105 analyser (Radiometer Medical A/S, Brenhej, Denmark). The glycosylated protein produced by the CHO cell line was quantified using an immunoassay and results were expressed as relative units. Specific productivity per day (pcd) was obtained from the slope of the linear regression of titers versus integrated viable cells.

Results

Glucose and Lactate Concentrations at Different Temperatures (FIG. 1)

Cells at a density of 0.6×10⁶ per ml were incubated in TCF25, in a serum-free medium containing 2.5 g/L of glucose, at 25, 29, 32, 34 or 37° C., in a humidified atmosphere of 5% CO₂ in air, for one to seven days. At all temperatures, there was little cell growth and cell density remained between 0.6 and 0.8×10⁶ cells/ml with a good viability for the first 4 to 5 days (data not shown).

The different cultures were tested for glucose consumption and lactate production. These parameters increased with increasing temperatures. As shown in FIG. 1, the glucose concentrations dropped rapidly below 0.5 g/L at the upper temperatures, on day 2 at 37° C., on day 3 at 34° C. and on day 4 at 32° C. The drop in glucose concentration correlated with an increase in the production of lactate to approximately 1.5 g/L. At 25 and 290C., the glucose consumption and the lactate production were very low: the glucose concentration remained above 1.5 g/L and lactate production below 0.25 g/L.

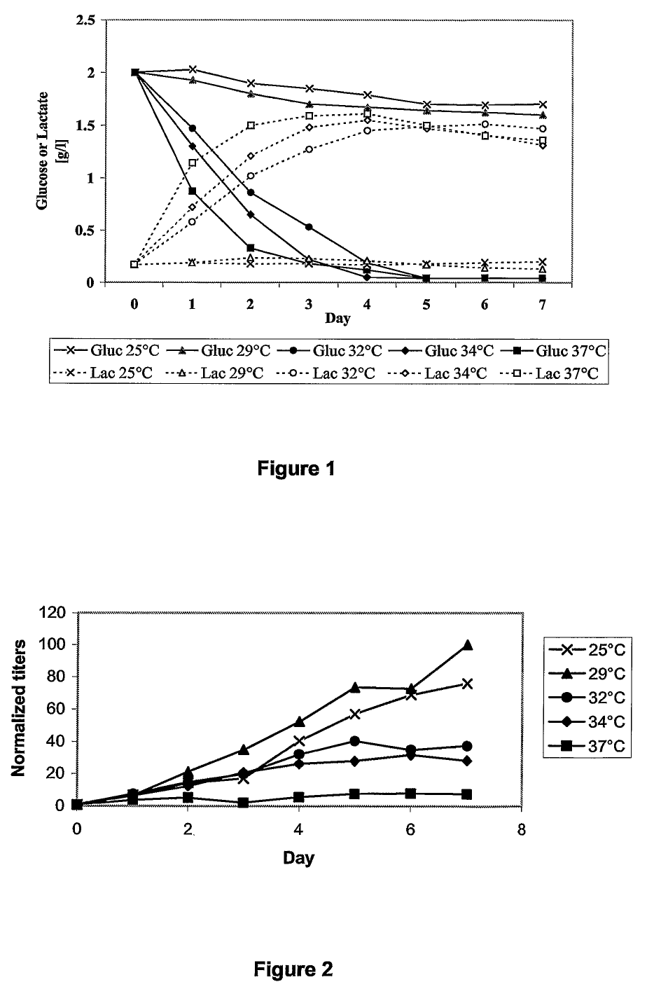

Titers of the TBP-1 at Different Temperatures (FIG. 2):

The amount of protein secreted per ml of medium was tested at each temperature. Titers were normalized by setting the maximum value to 100. As shown in FIG. 2, the titers decreased between 32 and 37° C., a temperature at which very little protein was secreted. A better productivity was obtained at 25 and 29° C., with best results at 29° C.

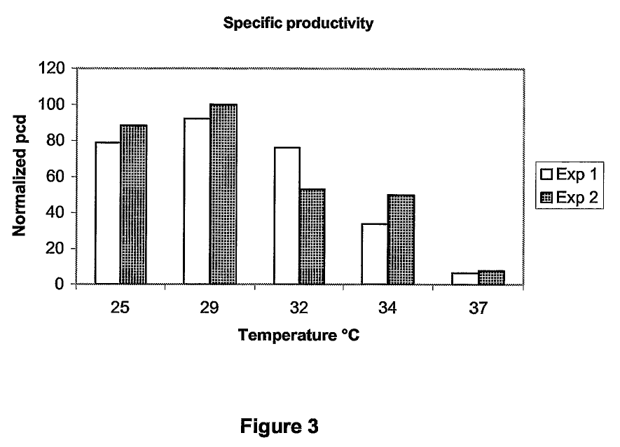

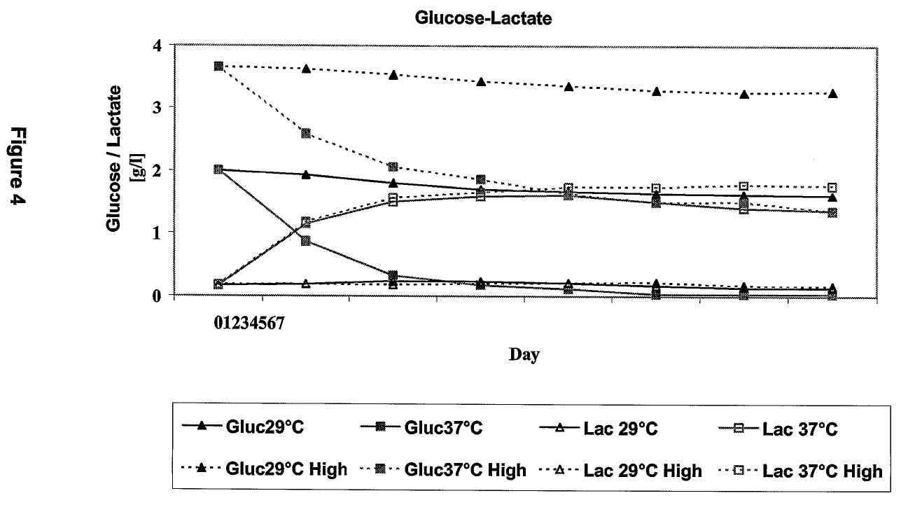

Specific Productivity (FIG. 3):

The specific productivity was analyzed taking into account the number of viable cells present in the culture. Setting the maximum value to 100 normalized the results. As shown in FIG. 3 for two experiments performed under the same conditions, the best specific productivity was obtained at 29° C., with values more than 10 fold higher than at 37° C.

Glucose and Lactate Concentrations as a Function of Time, in High (4 g/L) and Standard (2.5 g/L) Glucose Culture Medium

The previous experiments indicated that a higher productivity may be reached with lower temperatures. As glucose consumption and lactate production increased at higher temperatures and as glucose was rapidly depleted in cultures at 37° C., experiments were performed in order to verify that the low productivity observed at the higher temperatures was not due to a lack of nutrient (i.e. glucose) in the medium. For this purpose, 0.6×10⁶ cells/ml were incubated in TCF at 29 and 37° C. for one to seven days in serum-free medium, containing either 4 g/L of glucose (high glucose) or 2.5 g/L of glucose (standard glucose).

The glucose concentration in the medium had no effect on cell growth or viability (data not shown).

Glucose consumption and lactate production were high at 37° C. and low at 29° C. (FIG. 4). At 37° C., comparable amounts of glucose were consumed whatever initial glucose concentration in the medium, leading to levels below 0.5 g/L on day 2 in standard glucose medium, while in high glucose medium, the sugar concentration remained above or equal to 1.5 g/L up to day 6. At 29° C., glucose concentration remained above 3 g/L in cultures with high glucose. With standard glucose at 29° C., glucose concentrations between day 3 and day 7 were comparable to those obtained at 37° C. with high glucose (between 1.9 and 1.35 g/L).

Titers of the TBP-1 as a Function of Time, in High (4 g/L) and Standard Glucose (2.5 g/L) Culture Medium

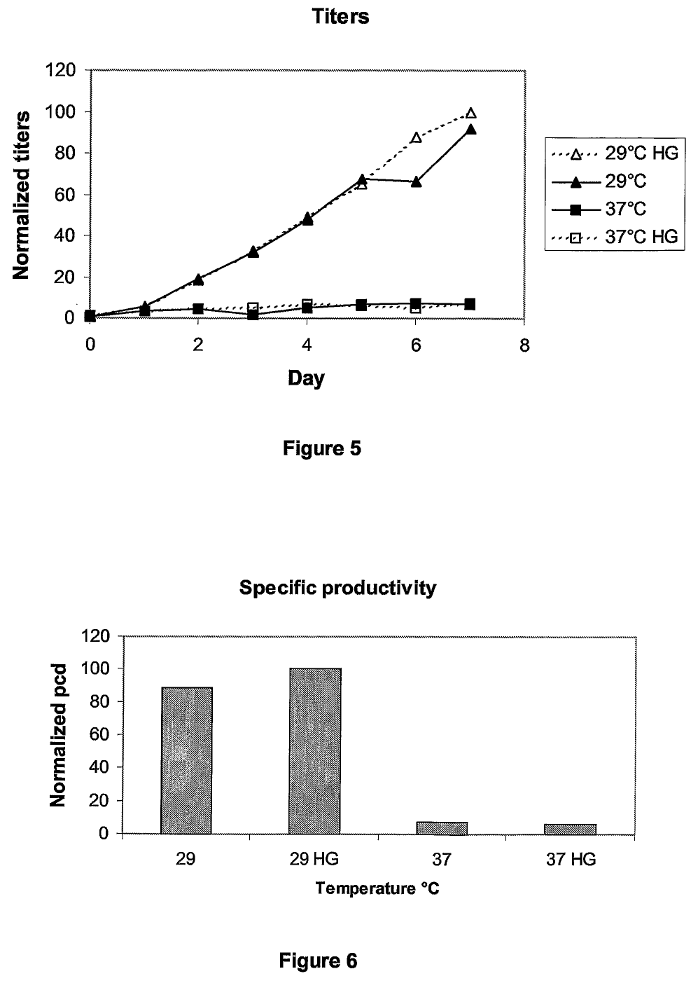

The amount of recombinant protein secreted in high glucose medium was not significantly different from that in standard glucose, as shown by titer measurements (FIG. 5). In both cases, the amount of protein produced was more than 10 times higher at 29° C. than at 37° C., although at 37° C. with high glucose containing medium, the remaining glucose concentration was comparable to that in the standard medium at 29° C. (˜1.5 g/L). This indicates that the low productivity observed at 37° C. was not due to the lack of glucose in the medium.

Specific Productivity of the TBP-1 as a Function of Time, in High (4g/L) and Standard (2.5 g/L) Glucose

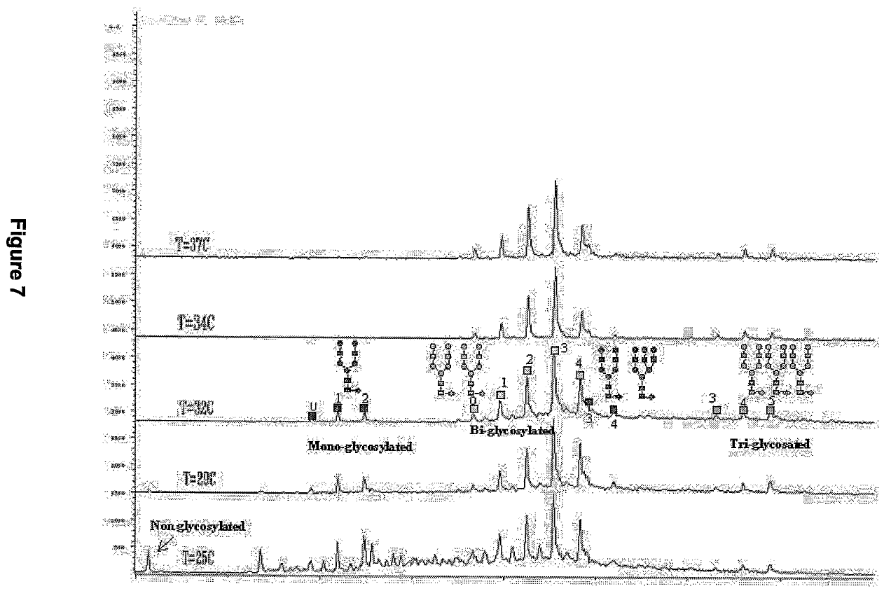

The specific productivity was very low at 37° C. in both standard and high glucose medium and was drastically increased at 29° C. (FIG. 6).

### Example 2

### Analysis of the Quality of the Molecule

Experimental Design

Experiments for the determination of the quality of the molecule by Mass Spectrometry were performed in TCF175. Triplicates were incubated at 25, 29, 32, 34 and 37° C. in medium with standard glucose (2.5 g/L) or high glucose (4 g/L) for seven days. Supernatants were then pooled, filtered on 0.8/0.2 μm filters and frozen at −70° C. before the TBP-1 was captured on an immobilized metal ion affinity chromatography column (IMAC).

The quality of the molecule in terms of glycosylation was tested on the partially purified protein by Mass spectrometry (MALDI-TOF) (Harvey, 1996). MALDI-TOF yields semi-quantitative information on the type and proportion of the individual oligosaccharide chains, allowing for example to determine which of the antennae are sialylated. TBP-1 has three putative N-linked glycosylation sites on asparagine residues and the main isoforms correspond to molecules with two glycosylation sites occupied. The glycans present on the molecule are of complex type, with a common core composed of 5 monosaccharides (2 N-acetylglucosamine & 3 Mannose). Different sugars (antenna) are added to this core structure, with sialic acids at their extremities. The number of sialic acids is variable, which contributes to the heterogeneity of the glycosylation. All the glycans are fucosylated and the main structure is a bi-antennary fucosylated species with a varying sialylation proportion.

Preparative Purification for Mass Spectrometry Analysis

A partial purification of the protein was necessary to enable the analysis by mass spectrometry. The frozen samples were thawed at 4° C. and then filtered on a 0.22 μm filter.

The filtrates were loaded onto an IMAC column. After elution, an aliquot containing 300-500 μg of the TBP-1 was analysed by mass spectrometry.

Mass Spectrometry

The method used was the MALDI-TOF MS (Matrix Assisted Laser Desorption Ionisation—Time-of-Flight Mass Spectrometry).

MALDI-TOF mass spectra were acquired on a Biflex II mass spectrometer (Bruker-Franzen Analytik GmBH, Brem, Germany) equipped with a 337-nm nitrogen laser, a reflectron and a delayed extraction system. The system was operated in the positive, linear ion mode. The matrix was a mixture of 2,6-dihydroxyacetophenone at a concentration of 10 mg/ml in acetonitrile/ethanol (50/50) and 1 M ammonium citrate (11/1, v/v). The analyte was mixed with the matrix (1/10, v/v) and deposited on the target. The mixture was allowed to dry at room temperature.

Determination of the S-Index

The S-index is an indicator of the overall sialylation level of the protein, computed from the analysis of the most abundant oligosaccharide species family (bi-glycosylated biantennary forms with 0 to 4 sialic acids).

The determination of the S-index is performed on the entire glycoprotein. It is calculated from the raw data spectrum from mass spectrometry (MALDI-TOF) considering the relative intensities of the ions of the main species:
A=Protein+2 Biantennary-Fucose 0 sialic acid
B=Protein+2 Biantennary-Fucose 1 sialic acid
C=Protein+2 Biantennary-Fucose 2 sialic acid
D=Protein+2 Biantennary-Fucose 3 sialic acid
E=Protein+2 Biantennary-Fucose 4 sialic acid

The S-index is defined as the sum of the relative intensities (pA, pB, pC, pD, pE=percent abundance of A, B, C., D, E) for each of these five species multiplied by the number of sialic acids:
S-Index=[(pA*0)+(pB*1)+(pC*2)+(pD*3)+(pE*4)]
Results

Mass Spectrometry

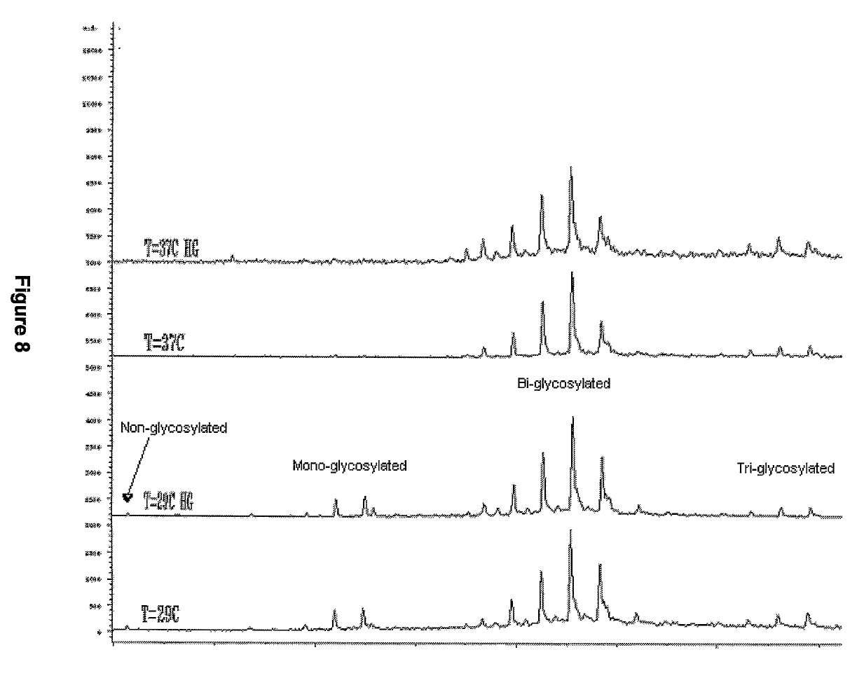

As shown in FIG. 7 and FIG. 8, the glycosylation of the molecule, when considering the most abundant species, which is bi-glycosylated bi-antennary, is all overall comparable at all temperatures (same degree of sialylation) and is not affected by different glucose concentrations in the medium. The same observation applies for the tri-glycosylated form. The mono-glycosylated form of the protein is favoured at lower temperatures and traces of the un-glycosylated form are detected.

Determination of the S-Index

These results were confirmed by the calculation from the raw data spectrum, of the S-index, which is an indicator of the overall sialylation level of the protein. As shown in Table 1, the S-index of all samples tested was comprised between 234 and 264.

TABLE 1
S-index values as a function of the temperature and glucose concentration.
HG = high glucose (i.e. 4 g/L); the other samples come from
cultures performed in a medium with 2.5 g/L of glucose.

| Temperature ° C. | S-index |
|---|---|
| 37° C. | 234 |
| 37° C. HG | 234 |
| 29° C. | 264 |
| 29° C. HG | 259 |
| 34° C. | 260 |
| 32° C. | 261 |
| 25° C. | 238 |

In conclusion, lowering the temperature from 37° C. to 29° C. had a beneficial effect on the productivity of recombinant CHO cells, increasing the amount of a secreted glycoprotein more than 10 fold without altering its quality in terms of glycosylation regarding the most abundant species (bi-glycosylated bi-antennary).

### Example 3

### TBP-1 Fed-Batch Production at 5L Scale

Experimental Design

A temperature study was performed comparing three different production temperatures in a fed-batch process at 5L scale where all other parameters were kept constant. Three runs were performed in a serum free culture medium with a growth phase at 37° C. and a production phase at 29° C. (Run1), 31° C. (Run2) and 34° C. (Run3).

From day 6 of culture, to day 22 or 24, each run was tested every other day for TBP-1 productivity.

The glycosylated protein produced by the CHO cell line was quantified using an immunoassay and results were expressed as normalized titers (the last value obtained at day 24 for run performed at 29° C. was set to 100).

The specific productivity was analyzed taking into account the number of viable cells present in the culture. Setting the maximum value to 100 normalized the results. The TBP-1 quality was assessed by the determination of the S-index using the method described in Example 2.

Results

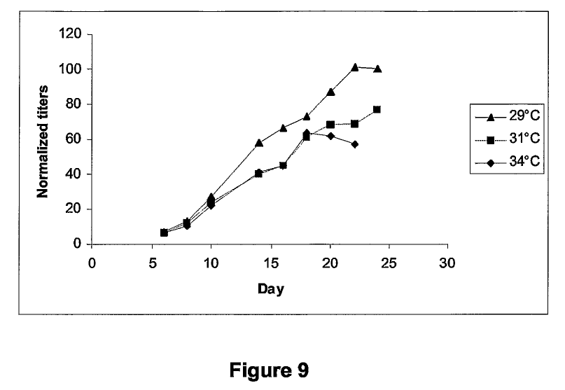

Titers of the TBP-1 at Different Temperatures (FIG. 9):

The titers obtained for the three runs performed in serum free culture medium are shown in FIG. 9. The titers are shown to increase with the temperature decrease. The best results were obtained at 29° C. where TBP-1 titer values became significantly higher at day 14 compared to the two experiments at higher temperature.

Specific Productivity

The specific productivity of TBP-1 producing cells at different temperatures was analysed taking into account the number of viable cells present in the culture. Viable cell density followed a similar trend at the three temperatures with the same decrease in viability that dropped below 40% between day 18 and 20 (data not shown). Specific productivity is shown in table 2 below. Setting the maximum value to 100 normalized the results.

TABLE 2

| | Production | Normalized specific |
|---|---|---|
| Run identity | temperature (° C.) | productivity |
| 1 | 29 | 100 |
| 2 | 31 | 85 |
| 3 | 34 | 69 |

The best specific productivity was obtained at 29° C. and was increased by 45% compared to that obtained at 34° C.

Determination of the S-index

TBP-1 quality data (S-index) after a capture step on Cu-chelating Sepharose FF column of samples harvested at day 21 are shown in Table 3.

TABLE 3

| Run ID | Eluate S-index |
|---|---|
| 1 (29° C.) | 196 |
| 2 (31° C.) | 199 |
| 3 (34° C.) | 214 |

### REFERENCES

Altschul, S. F., Gish, W., Miller, W., Myers, E. W., and Lipman, D. J. (1990). Basic local alignment search tool. J. Mol. Biol. 215, 403-410.
Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res. 25, 3389-3402.
Andersen, D. C., Bridges, T., Gawlitzek, M., and Hoy, C. (2000). Multiple cell culture factors can affect the glycosylation of Asn-184 in CHO-produced tissue-type plasminogen activator. Biotechnol. Bioeng. 70, 25-31.
Barnabé N. and Butler, M. (1994). Effect of temperature on nucleotide pools and monoclonal antibody production in a mouse hybridoma. Biotechnol. Bioeng. 44, 1235-1245.
Borys, M. C., Linzer, D. I. H., and Papoutsakis, E. T. (1993). Culture pH affects expression rates and glycosylation of recombinant mouse placental lactogen proteins by Chinese hamster ovary (CHO) cells. Biotechnology (N.Y.) 11, 720-724.
Castro, P. M., Ison, A. P., Hayter, P. M., and Bull, A. T. (1995). The macroheterogeneity of recombinant human interferon-gamma produced by Chinese-hamster ovary cells is affected by the protein and lipid content of the culture medium. Biotechnol. Appl. Biochem 21 (Pt 1), 87-100.
Chuppa, S., Tsai, Y.-S., Yoon, S., Shackleford, S., Rozales, C., Bhat, R., Tsay, G., Matanguihan, C., Konstantinov, K., and Naveh, D. (1997). Fermentor temperature as a tool for control of high-density perfusion cultures of mammalian cells. Biotechnol. Bioeng. 55, 328-338.
Devereux, J., Haeberli, P., and Smithies, O. (1984). A comprehensive set of sequence analysis programs for the VAX. Nucleic Acids Res. 12, 387-395.
Ducommun, P., Ruffieux, P. A., Kadouri, A., von Stockar, U., and Marison, I. W. (2002). Monitoring of temperature effects on animal cell metabolism in a packed bed process. Biotechnol. Bioeng. 77, 838-842.
Furukawa, K. and Ohsuye, K. (1998). Effect of culture temperature on a recombinant CHO cell line producing a C-terminal α-amidating enzyme. Cytotechnology 26, 153-164.
Furukawa, K. and Ohsuye, K. (1999). Enhancement of productivity of recombinant α-amidating enzyme by low temperature culture. Cytotechnology 31, 85-94.
Gawlitzek, M., Ryll, T., Lofgren, J., and Sliwkowski, M. B. (2000). Ammonium alters N-glycan structures of recombinant TNFR-lgG: degradative versus biosynthetic mechanisms. Biotechnol. Bioeng. 68, 637-646.
Goldman, M. H., James, D. C., Ison, A. P., and Bull, A. T. (1997). Monitoring proteolysis of recombinant human interferon-gamma during batch culture of Chinese hamster ovary cells. Cytotechnology 23, 103-111.
Grantham, R. (1974). Amino acid difference formula to help explain protein evolution. Science 185, 862-864.
Harvey, D. J. (1996). Matrix-assisted laser desorption/ionisation mass spectrometry of oligosaccharides and glycoconjugates. J Chromatogr. A 720, 429-446.
Hendrick, V., Winnepenninckx, P., Abdelkafi, C., Vandeputte, O., Cherlet, M., Marique, T., Renemann, G., Loa, A., Kretzmer, G., and Werenne, J. (2003). Increased productivity of recombinant tissular plasminogen activator (t-PA) by butyrate and shift of temperature : a cell cycle phases analyses. Cytotechnology 36, 71-83.
Hirschberg, C. B. and Snider, M. D. (1987). Topography of glycosylation in the rough endoplasmic reticulum and Golgi apparatus. Annu Rev Biochem 56, 63-87.
Jenkins, N., Parekh, R. B., and James, D. C. (1996). Getting the glycosylation right: implications for the biotechnology industry. Nat Biotechnol. 14, 975-981.
Kaufmann, H., Mazur, X., Fussenegger, M., and Bailey, J. E. (1999). Influence of low temperature on productivity, proteome and protein phosphorylation of CHO cells. Biotechnol. Bioeng. 63, 573-582.
Kaufmann, H., Mazur, X., Marone, R., Bailey, J. E., and Fussenegger, M. (2001). Comparative analysis of two controlled proliferation strategies regarding product quality, influence on tetracycline-regulated gene expression, and productivity. Biotechnol. Bioeng. 72, 592-602.
Loetscher, H., Pan, Y. C., Lahm, H. W., Gentz, R., Brockhaus, M., Tabuchi, H., and Lesslauer, W. (1990). Molecular cloning and expression of the human 55 kd tumor necrosis factor receptor. Cell 61, 351-359.
Moore, A., Mercer, J., Dutina, G., Donahue, C. J., Bauer, K. D., Mather, J. P., Etcheverry, T., and Ryll, T. (1997). Effects of temperature shift on cell cycle, apoptosis and nucleotide pools in CHO cell batch cultures. Cytotechnology 23, 47-54.
Mueller, P. P., Schlenke, P., Nimtz, M., Conradt, H. S., and Hauser, H. (1999). Recombinant glycoprotein product quality in proliferation-controlled BHK-21 cells. Biotechnol. Bioeng. 65, 529-536.
Munzert, E., Mthing, J., Buntemeyer, H., and Lehmann, J. (1996). Sialidase activity in culture fluid of Chinese hamster ovary cells during batch culture and its effects on recombinant human antithrombin III integrity. Biotechnol. Prog. 12, 559-563.
Nophar, Y., Kemper, O., Brakebusch, C., Englemann, H., Zwang, R., Aderka, D., Holtmann, H., and Wallach,D. (1990). Soluble forms of tumor necrosis factor receptors (TNF-Rs). The cDNA for the type I TNF-R, cloned using amino acid sequence data of its soluble form, encodes both the cell surface and a soluble form of the receptor. EMBO J 9, 3269-3278.
Nyberg, G. B., Balcarcel, R. R., Follstad, B. D., Stephanopoulos, G., and Wang, D. I. (1999). Metabolic effects on recombinant interferon-gamma glycosylation in continuous culture of Chinese hamster ovary cells. Biotechnol. Bioeng. 62, 336-347.
Pearson, W. R. (1990). Rapid and sensitive sequence comparison with FASTP and FASTA. Methods Enzymol. 183, 63-98.
Pearson, W. R. and Lipman, D. J. (1988). Improved tools for biological sequence comparison. Proc. Natl. Acad. Sci. U.S.A. 85, 2444-2448.
Schall, T. J., Lewis, M., Koller, K. J., Lee, A., Rice, G. C., Wong, G. H., Gatanaga, T., Granger, G. A., Lentz, R., Raab, H., and . (1990). Molecular cloning and expression of a receptor for human tumor necrosis factor. Cell 61, 361-370.
Smith, C. A., Davis, T., Anderson, D., Solam, L., Beckmann, M. P., Jerzy, R., Dower, S. K., Cosman, D., and Goodwin, R. G. (1990). A receptor for tumor necrosis factor defines an unusual family of cellular and viral proteins. Science 248, 1019-1023.
Smith, T. F. and Waterman, M. S. (1981). Identification of common molecular subsequences. J. Mol. Biol. 147, 195-197.
Sureshkumar, G. K. and Mutharasan, R. (1991). The influence of temperature on a mouse-mouse hybridoma growth and monoclonal antibody production. Biotechnol Bioeng. 37, 292-295.
Weidemann, R., Ludwig, A., and Kretzmer, G. (1994). Low temperature cultivation—a step towards process optimisation. Cytotechnology 15, 111-116.
Werner, R. G., Noe, W., Kopp, K., and Schluter, M. (1998). Appropriate mammalian expression systems for biopharmaceuticals. Arzneimiftelforschung. 48, 870-880.
Yang, M. and Butler, M. (2000). Effects of ammonia on CHO cell growth, erythropoietin production, and glycosylation. Biotechnol. Bioeng. 68, 370-380.
Yoon, S. K., Song, J. Y., and Lee, G. M. (2003). Effect of low culture temperature on specific productivity, transcription level, and heterogeneity of erythropoietin in Chinese hamster ovary cells. Biotechnol Bioeng. 82, 289-298.

1-16. (canceled)

17. A method for producing a recombinant polypeptide comprising culturing a mammalian cell line, the cell line expressing a recombinant polypeptide in a production phase at a temperature at or below 29° C.

18. The method of claim 17, wherein the polypeptide is a Tumor Necrosis Factor Binding Protein (TBP), or a mutein or fragment thereof.

19. The method of claim 18, wherein the polypeptide is recombinant human TBP-1 or TBP-2.

20. The method of claim 19, wherein the polypeptide is expressed by a mammalian cell line cornp rising a DNA sequence encoding a TBP-1 polypeptide selected from the group consisting of:
(a) a polypeptide comprising SEQ ID NO: 1;
(b) a mutein of (a), wherein the amino acid sequence has at least 40% or 50% or 60% or 70% or 80% or 90% identity to the sequence in (a);
(c) a mutein of (a) which is encoded by a DNA sequence, which hybridizes to the complement of the native DNA sequence encoding (a) under moderately stringent conditions or under highly stringent conditions;
(d) a mutein of (a) wherein any changes in the amino acid sequence are conservative amino acid substitutions to the amino acid sequences in (a); and
(e) a salt or an isoform, fused protein, functional derivative, active fraction or circularly permutated derivative of (a).

21. The method of claim 19, wherein the polypeptide is expressed by a mammalian cell line comprising a DNA sequence encoding a TBP-2 polypeptide selected from the group consisting of:
(a) a polypeptide comprising SEQ ID NO: 2;
(b) a mutein of (a), wherein the amino acid sequence has at least 40% or 50% or 60% or 70% or 80% or 90% identity to the sequence in (a);
(c) a mutein of (a) which is encoded by a DNA sequence, which hybridizes to the complemerit of the native DNA sequence encoding (a) under moderately stringent conditions or under highly stringent conditions;
(d) a mutein of (a) wherein any changes in the amino acid sequence are conservative amino acid substitutions to the amino acid sequences in (a);
(e) a salt or an isoform, fused protein, functional derivative, active fraction or circularly permutated derivative of (a).

22. The method of claim 20, wherein the mammalian cell line is cultured at a temperature between 20° C. and 29° C.

23. The method of claim 21, wherein the mammalian cell line is cultured at a temperature between 20° C. and 29° C.

24. The method of claim 22, wherein the mammalian cell line is cultured at a temperature of about 25 to 29° C.

25. The method of claim 24, wherein the mammalian cell line is cultured at a temperature of about 26° C., or about 27° C., or about 28° C.

26. The method of claim 24, wherein the mammalian cell line is cultured at a temperature of about 29° C.

27. The method of claim 23, wherein the mammalian cell line is cultured at a temperature of about 25 to 29° C.

28. The method of claim 27, wherein the mammalian cell line is cultured at a temperature of about 26° C., or about 27° C., or about 28° C.

29. The method of claim 27, wherein the mammalian cell line is cultured at a temperature of about 29° C.

30. The method of claim 17, wherein the mammalian cell line is a CHO cell line.

31. The method of claim 17, wherein the medium used during the production phase is serum free.

32. The method of claim 17, further comprising collecting the polypeptide from the medium.

33. The method of claim 17, further comprising purifying the polypeptide from medium or cell derived components.

34. The method of claim 17, further comprising formulating the purified polypeptide with a pharmaceutically acceptable carrier.

35. An isolated polypeptide produced by the method of claim 17, said polypeptide being mono-glycosylated.
